(19) United States
(12) Patent Application Publication
(10) Pub. No.: US 2024/0324996 A1
(43) Pub. Date: Oct. 3, 2024

(54) FLEX INTERCONNECT FOR A TRANSDUCER ARRAY ON AN INTRACARDIAC ECHOCARDIOGRAPHY CATHETER DEVICE

(71) Applicant: yoR Labs, Inc., Beaverton, OR (US)

(72) Inventors: Dakota Pellegrino, Hillsboro, OR (US); Jessica Myrtle Chapman, Beaverton, OR (US); Anshumali Roy, Portland, OR (US)

(21) Appl. No.: 18/612,957

(22) Filed: Mar. 21, 2024

Related U.S. Application Data
(60) Provisional application No. 63/492,424, filed on Mar. 27, 2023.

(51) Int. Cl. A61B 8/00; A61B 8/08; A61B 8/12

(52) U.S. Cl. CPC A61B 8/4494; A61B 8/12; A61B 8/0883

(57) ABSTRACT

An intracardiac echocardiography (“ICE”) catheter device including a flexible circuit sized for insertion into a vessel of a patient. The flexible circuit can have a transducer array positioned on the outer surface of the flexible circuit at a proximal region. A cable connection portion can be positioned at a distal region of the flexible circuit, where a plurality of traces can couple the transducer array to the cable connection portion. The arrangement of the plurality of traces, which couple the transducer array and the cable connection portion, can improve the signal processing time of the ICE catheter device which can result in better imaging.

### INCORPORATION BY REFERENCE TO ANY PRIORITY APPLICATIONS

This application claims the benefit of U.S. Provisional Application No. 63/492,424, filed Mar. 27, 2023, which is incorporated by reference herein in its entirety. Any and all applications for which a foreign or domestic priority claim is identified in the Application Data Sheet as filed with the present application are hereby incorporated by reference under 37 CFR 1.57 for all purposes and for all that they contain.

### TECHNICAL FIELD

The present disclosure relates to a flex interconnect for a transducer array on an intracardiac echocardiography (“ICE”) catheter.

### BACKGROUND

Intracardiac echocardiography (“ICE”) catheters are widely used for diagnosing or assessing injured or diseased tissues or vessels, such as an artery within the human body to determine if further treatment is needed. As the ICE catheter passes through the tissue or vessel, an ultrasonic transducer array disposed on the surface of the catheter can emit ultrasonic pulses and/or signals which images the tissues or vessels. To ensure the ICE catheter can effectively navigate the tissues and/or vessels, a flexible circuit or flexible interconnect having a transducer array disposed on the surface of the flexible circuit can enhance imaging of the affected tissues and/or vessels.

### SUMMARY

Embodiments of devices and methods for a flex interconnect to a transducer array on an ICE catheter are disclosed.

One innovation includes an intracardiac echocardiography (“ICE”) catheter device having a flexible circuit sized for insertion into a vessel of a patient. The flexible circuit can have an outer surface and an inner surface configured to face an opposite direction of the outer surface. The flexible circuit can have a distal region, a proximal region having a plurality of traces, and a transducer array positioned on the outer surface of the proximal region and electrically coupled to the plurality of traces. The plurality of traces can be arranged to electrically couple to a plurality of transducers of the transducer array. The flexible circuit can have a backing block on the inner surface opposite the transducer array. The flexible circuit can have a cable connection portion in the distal region of the flexible circuit. The cable connection portion can have one or more connectors, where one or more bundles can be positioned on the outer surface of the flexible circuit and can be coupled to the one or more connectors in the cable connection portion. In some embodiments, each bundle can include a plurality of communication channels. The cable connection portion can be positioned such that a cable connection portion inner surface faces the backing block, and the backing block can be between the transducer array and the cable connection portion.

Various embodiments can include one or more additional features, and different features. In some embodiments disclosed herein, the flexible circuit can have a shield located on a portion of the flexible circuit such that the cable connection portion is between the transducer array and the shield. In some embodiments, the shield can be adjacent to the cable connection portion. The shield can be folded behind the cable connection portion and the outer surface of the shield can face the cable connection portion. In some embodiments, the plurality of transducers can be sixty-four transducers. In some embodiments, the plurality of transducers can be coupled to the plurality of traces, where the plurality of traces can be sixty-four traces. In some embodiments, the one or more connectors can be four connectors. In some embodiments, one of the one or more connectors can be coupled to sixteen of the plurality of traces. In some embodiments, the one or more bundles can be sixteen bundles. In some embodiments, one of the one or more connectors can be coupled to four of the one or more bundles.

In some embodiments disclosed herein, the flexible circuit transducer array is positioned a distance from a cut line, the cut line being indicative of a place on the flexible circuit that can be cut to separate a test connection portion from the transducer array, and wires (e.g., bundles) connected to the transducer array (and a shield for embodiments that include a shield). In some embodiments, the distance from the cut line to the transducer array can be 50 micrometers. In some embodiments, the flexible circuit can have a test connection portion, where the test connection portion can be located on one side of the cut line and the transducer array can be located on an opposite side of the cut line. In some embodiments, the test connection portion can have an electrical connection to each of the plurality of transducers. In some embodiments, the plurality of traces can have a thickness of between about 0.02 mm and about 0.09 mm, for example, a thickness of 0.02 mm. 0.03 mm. 0.04 mm, 0.05 mm, 0.06 mm, 0.07 mm, 0.08 mm, or 0.09 mm, plus or minus 0.005 mm. For example, in some preferred embodiments the plurality of traces has a thickness of about 0.05 mm. In some embodiments, the plurality of traces can have a length, between the transducer array and the connectors where the traces are connected to a communication channel for providing signals to the transducer array and receiving signals from the transducer array, of between about 1.40 mm and about 3.43 mm. The plurality of traces can be made of copper or any suitable conductive material. In some embodiments, the shield can have a length between about 23 mm and about 27 mm. In some embodiments, the shield can have a width between about 30 mm to 80 mm. In some embodiments, the shield can have a width of between about 50 mm and about 60 mm. In some embodiments, the shield can have one or more cutouts. The shield can be made of copper or another suitable material, for example, a material that provides electromagnetic shielding. In some embodiments, the flexible circuit can have a ground bar. The ground bar may be arranged to be at least partially around the plurality of transducers and the one or more connectors. The plurality of traces can be routed to, and connect to, a connector portion (“connector”) in various configurations. In some embodiments, a subset of the plurality of traces can connect to a left portion of the one or more connectors. In some embodiments, a subset of the plurality of traces can connect to a right portion of the one or more connectors. In some embodiments, a length between the transducer array and the cable connection portion can be between about 0.85 mm and about 0.95 mm. In some embodiments, the cable connection portion can have a length of approximately 2 mm. In some embodiments, an arraignment of the plurality of traces can reduce a signal processing time from the one or more bundles to the plurality of transducers. In some embodiments, one of the one or more bundles can have a diameter of approximately 185 micrometers. For example, 185 micrometers plus or minus 30 micrometers, plus or minus 20 micrometers, or plus or minus 10 micrometers. In some embodiments, the plurality of traces can extend in a non-linear path from the transducer array to the one or more connectors. The non-linear path can improve a signal processing time for the device.

In some embodiments, the techniques described herein can relate to a method of assembly of circuit for an intracardiac echocardiography (“ICE”) catheter device, which can include obtaining a flexible circuit having an outer surface and an inner surface configured to face an opposite direction of the outer surface. The flexible circuit can have a proximal portion which can couple to a transducer array, where the proximal portion can have a plurality of traces arranged to electrically couple to a plurality of transducers of the transducer array. The flexible circuit can have a distal portion, which can have a cable connection portion. The cable connection portion can have one or more connectors electrically connected to the plurality of traces. The device can have a test connection area which can include an electrical connection to each of the plurality of traces. The method can include coupling the transducer array to the plurality of traces in the proximal portion. After testing an electrical connection between the transducer array and the plurality of traces using the test connection area, a user can separate the test connection area from the proximal portion of the flexible circuit. The method can include coupling one or more bundles positioned on the outer surface to the one or more connectors in the cable connection portion, where each bundle can have a plurality of communication channels.

In some embodiments, the method can include positioning a backing block on the inner surface opposite the transducer array.

In some embodiments, the method can include positioning the cable connection portion such that the inner surface of the cable connection portion faces the backing block.

In some embodiments, the method can include positioning the cable connection portion such that the inner surface of the cable connection portion can face the backing block, which can include folding a distal region of the flexible circuit underneath the transducer array.

In some embodiments of the method disclosed herein, the flexible circuit can have a shield which can be located on a portion of the flexible circuit such that the cable connection portion is between the transducer array and the shield.

In some embodiments, the method can include positioning a backing block on the inner surface opposite the transducer array.

In some embodiments, the techniques described herein relate to a method, further including positioning the cable connection portion such that the inner surface of the cable connection portion faces the backing block.

In some embodiments, the method can include positioning the cable connection portion such that the inner surface of the cable connection portion can face the backing block. The method can include folding a distal region of the flexible circuit underneath the transducer array.

In some embodiments, the method can include positioning the shield adjacent to the cable connection portion where the shield can be folded behind the cable connection portion such that the outer surface of the shield faces the cable connection portion.

### BRIEF DESCRIPTION OF THE DRAWINGS

The features and advantages of the embodiments described herein will become more fully apparent from the following description and appended claims, taken in conjunction with the accompanying drawings. These drawings may depict only certain embodiments in accordance with the disclosure and are not to be considered limiting of its scope. In the drawings, similar reference numbers or symbols typically identify similar components, unless context dictates otherwise. In some instances, the drawings may not be drawn to scale.

FIG. 1A illustrates a top view of a portion of an intracardiac echocardiography (“ICE”) catheter device 100 that includes a test connection area, a flexible circuit and a transducer array shown in a flat configuration.

FIG. 1B illustrates a partial top view of a distal portion of the ICE catheter device.

FIGS. 2A and 2B illustrate an end/rolled side view of a schematic of the ICE catheter device of FIG. 1A, where FIG. 2A is an end view and FIG. 2B is a perspective view.

FIG. 3A illustrates a top view of another embodiment of a portion of an ICE catheter device illustrated in a flat configuration.

FIG. 3B illustrates a partial top view of a distal area of the ICE catheter device of FIG. 3A that includes a shield, illustrated in a flat configuration.

FIGS. 4A and 4B illustrate an end/rolled side view of a schematic of the ICE catheter device of FIG. 3A, where FIG. 4A is an end view and FIG. 4B is a perspective view.

FIG. 5A illustrates a top perspective view of another embodiment of the ICE catheter device shown in a flat configuration.

FIG. 5B illustrates a partial top perspective view of a distal area of the intracardiac echocardiography (“ICE”) catheter device of FIG. 5A in a flat configuration.

FIG. 6 illustrates a partial top view of the intracardiac echocardiography (“ICE”) catheter device of FIG. 3A in a flat configuration.

FIG. 7 illustrates an example of a portion of the ICE catheter device of FIG. 1A positioned on an insertion device (e.g., a catheter).

### DETAILED DESCRIPTION

The following detailed description describes embodiments generally relating to flex interconnects to a transducer array that can be used on a catheter, e.g., an intracardiac echocardiography (“ICE”) catheter, some of which are illustrated in the figures. These embodiments are not intended to be limiting, and various modifications, variations, combinations, etc., of the features of these embodiments are possible and within the scope of this disclosure.

Intracardiac echocardiography (ICE) uses sound waves to produce images of the heart. During intracardiac echocardiography, a tiny catheter with an ultrasound sensor is passed into the heart where images of the heart can be captured. Intracardiac echocardiography has become an integral part of a variety of percutaneous interventional and electrophysiology procedures. ICE catheter systems can have a sensor array positioned near a distal end of a catheter, for example, an array of ultrasound transducers, or doppler ultrasound transducers, arranged coupled to a surface of the distal tip of an ICE catheter. The transducers are configured to emit ultrasonic energy and receive signals generated (e.g., by reflection) by the emitted energy, and provide the received signals to an ICE catheter processing system to create an image of tissue or vessels being imaged.

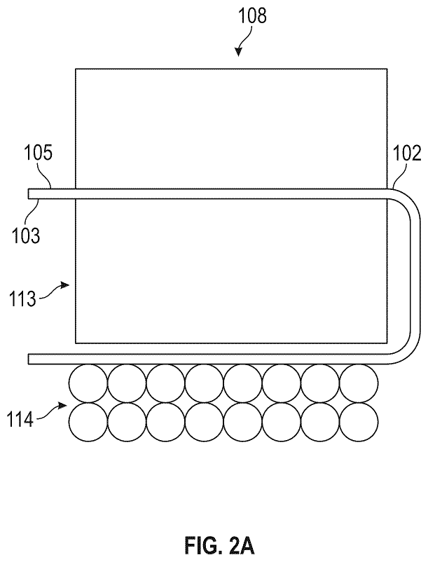

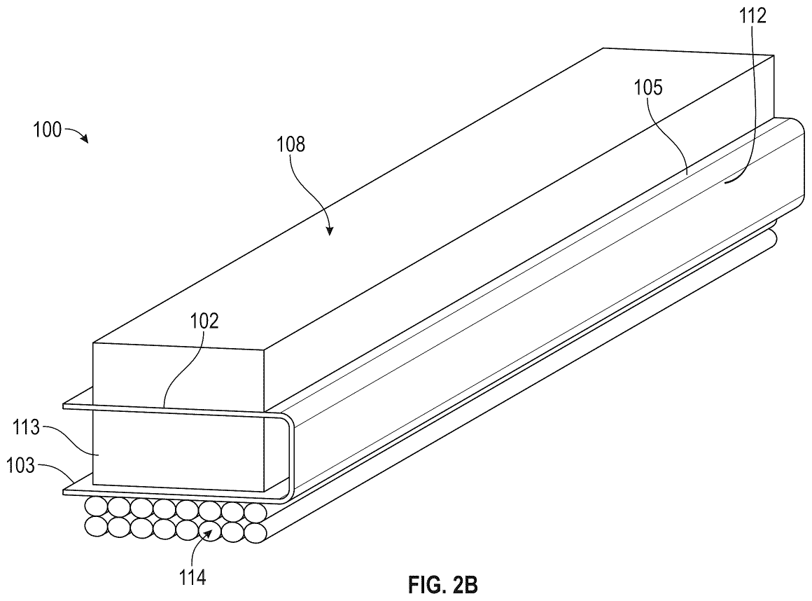

In embodiments described herein, a flexible circuit can be included in an ICE catheter device. The flexible circuit can include a sensor assembly that includes a flexible member having electrical communication channels configured to provide ultrasonic drive signals to a transducer array and communicate information received from the transducer array to a processing system for testing of the transducer array, e.g., from a cable connection portion on a distal end of the flexible circuit. The flexible member can include electrical communication channels configured to provide ultrasonic drive signals to a transducer array and communicate information received from the transducer array to a processing system when the flexible circuit is incorporated into a catheter, e.g, on a proximal end of the flexible circuit. The flexible circuit can include a transducer array coupled to the flexible member. At least of portion of the communication channels can be on the flexible member (for example, as illustrated in FIGS. 2A and 2B). The transducer array can be coupled to the flexible member during manufacturing. The flexible circuit which can be sized and shaped for insertion into a vessel or a tissue structure. In an example, a flexible circuit includes several layers including a base layer, a conductive layer, and a cover layer. The conductive layer can be copper or another conductive material. A flexible circuit can also include an adhesive layer. The flexible circuit can have a distal region and a proximal region, where the proximal region can include an array of transducers (e.g., transducer array), for example, arranged on a chip. Furthermore, by having a flexible circuit instead of a rigid circuit, the imaging capabilities of the ICE catheter device can improve, and the signals received and processed by the ICE catheter device can better determine if there are any irregularities in the vessel or tissue structure (e.g., blood flow circulation problems, blood flow stoppage, etc.). The distal region of the ICE catheter device can have a cable connection portion, where the signals which are emitted and received by the transducer array can be received/sent to image the vessel where the transducer array is placed. In some embodiments, the signals can be relayed from the transducer array to the cable connection portion via a plurality of traces, where the plurality of traces can be a conductive material (e.g., copper). Furthermore, the plurality of traces can be arranged on the flexible circuit disposed between the cable connection portion and the transducer array.

Advantageously, the size and arrangement of the plurality of traces can allow the signals emitted and received by the transducer array to have a shorter relay time between the transducer array and cable connection area (e.g., due to shorter distances the signals travel) which can improve signal processing, signal processing time, and imaging.

In some embodiments, the flexible circuit (and ICE catheter devices) described herein can have a shield structure (“shield”). The shield can be configured to partially protect or completely protect the cable connection portion. When the flexible circuit is flat, the shield can be proximal to the cable connection portion, that is, positioned such that the cable connection portion is between the shield and the test connection area. Additionally, when in a rolled configuration the shield can be located in a position to protect a lateral portion of the cable connection portion.

Embodiments of a flex interconnects (e.g., flexible circuits) for a transducer array on an ICE catheter are described herein. The following is a list of certain components that are described and enumerated in this disclosure in reference to the above-listed figures. Other components, or aspects of these components, may not be included in the list but are disclosed in the figures and description. Accordingly, any aspect illustrated in the figures, whether or not called out separately herein, can form a portion of various embodiments and may provide basis for claim limitation relating to such aspects, with or without additional description. Certain enumerated components include:

| FIG. No. | Figure Description |
|---|---|
| 100 | Portion of a catheter (e.g., an ICE catheter) |
| 101 | Test connection area |
| 102 | Outer surface |
| 103 | Inner surface |
| 104 | Distal region |
| 105 | Flexible circuit |
| 106 | Proximal region |
| 108 | Transducer array |
| 109 | Plurality of transducers |
| 110 | Cable connection portion |
| 111 | One or more connectors (“connectors”) |
| 112 | Plurality of traces (“traces”) |
| 113 | Backing blocks |
| 114 | One or more bundles (“bundles”) |
| 116 | One or more cutouts (“cutouts”) |
| 117 | Distance from transducer array to the cut line |
| 118 | Length between transducer array and cable connection portion |
| 119 | Length of cable connection portion |
| 120 | Ground bar |
| 121 | Cut line |
| 122 | Plurality of electrical connectors |
| 125 | Cable connection portion width |
| 126 | Distal end of the plurality of transducers |
| 127 | Proximal end of the plurality of transducers |
| 140 | Insertion device |
| 150 | Distal end of flexible circuit |
| 160 | Proximal end of flexible circuit |
| 300 | Intracardiac echocardiography catheter |
| 301 | Test connection area |
| 302 | Outer surface |
| 303 | Inner surface |
| 304 | Distal region |
| 305 | Flexible circuit |
| 306 | Proximal region |
| 308 | Transducer array |
| 309 | Plurality of transducers |
| 310 | Cable connection portion |
| 311 | One or more connectors |
| 312 | Plurality of traces |
| 313 | Backing blocks |
| 314 | One or more bundles |
| 316 | One or more cutouts |
| 317 | Distance from transducer array to the cut line |
| 318 | Length between transducer array and cable connection portion |
| 319 | Length of cable connection portion |
| 320 | Ground bar |
| 321 | Cut line |
| 322 | Plurality of electrical connectors |
| 323 | Shield |
| 324 | Shield length |
| 325 | Shield width |
| 326 | Distal end of the plurality of transducers |
| 327 | Proximal end of plurality of transducers |

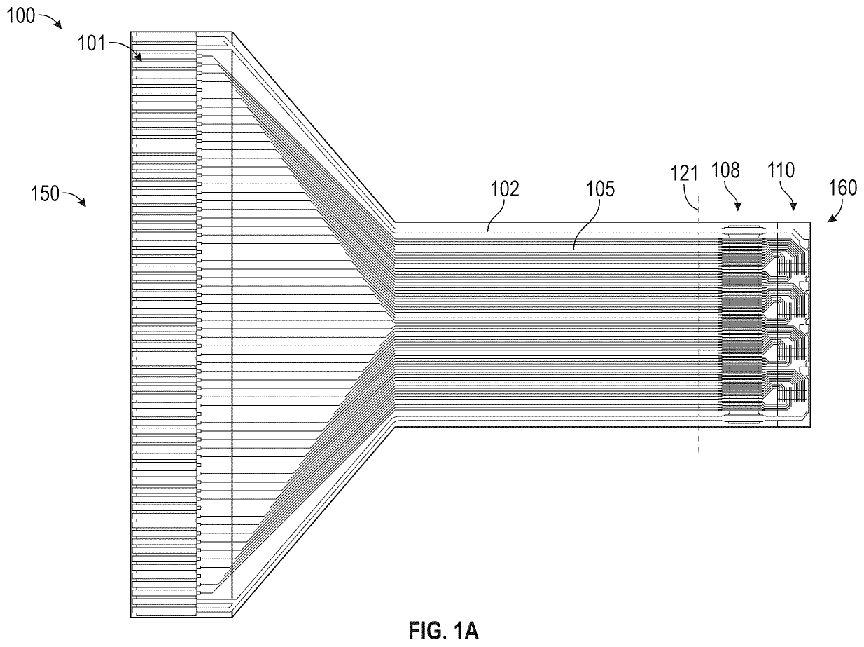

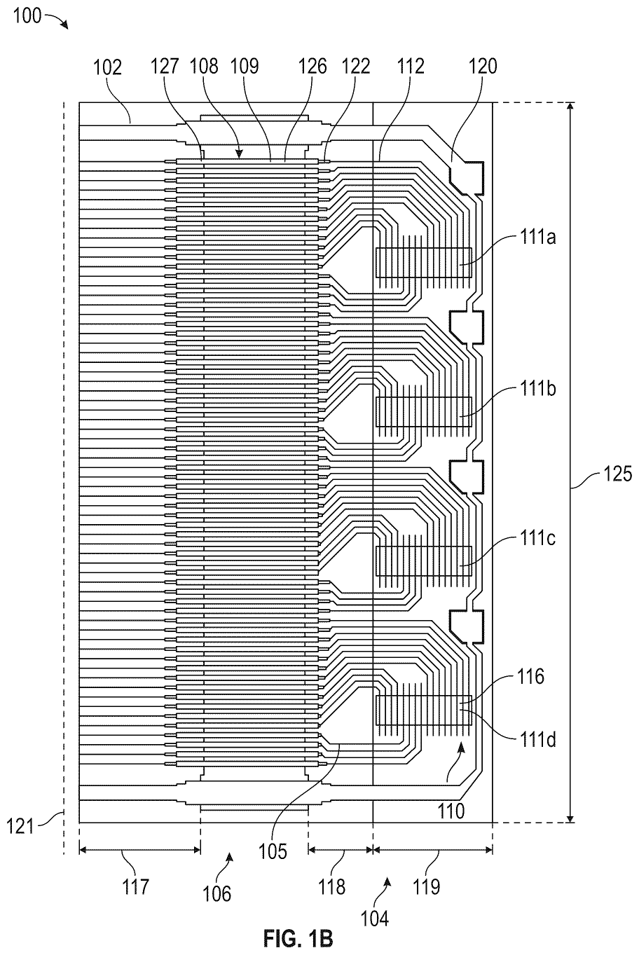

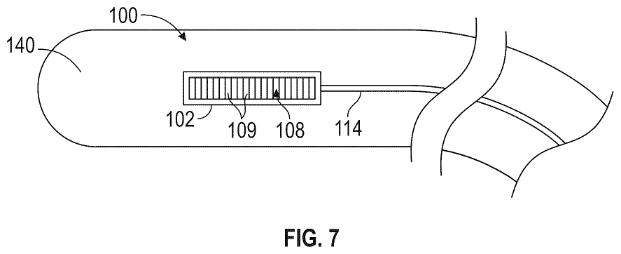

FIG. 1A illustrates top view of an example of a portion of an intracardiac echocardiography (“ICE”) catheter device 100 in a flat configuration, according to an embodiment of the present disclosure. The illustrated portion of the ICE catheter device 100 (which may be referred to herein as “ICE catheter device 100” or “ICE catheter circuit 100” for ease of reference) can include a transducer array 108 connected to proximal end of a flexible circuit 105, a test connection area 101 connected to the flexible circuit 105 distal end 150 of the, and a cable connection portion 110 on the flexible circuit 105 proximal side 160 connected to the transducer array 108. Although embodiments are referred to herein as indicating association with an ICE catheter or ICE catheter circuit, the embodiments described herein can be used for other catheters as well which have other functionality (e.g., intravascular ultrasound (IVUS)). The transducer array 108 and the cable connection portion 110 can be included as part of an ICE catheter, as illustrated in FIG. 7. The ICE catheter circuit 100 in FIG. 1 is an example of certain components in a configuration to facilitate testing of the transducer array 108 (e.g., the transducer array 108 itself, the connection of the transducer array 108 to the flexible circuit, etc.) before it is incorporated into a catheter. After testing of the transducer array 108, the test connection area 101 can be separated from the transducer array 108 and the portion of the flexible circuit the transducer array is attached to. In use, the transducer array 108 can receive signals from the cable connection 110 and emit ultrasound signals. The transducer array 108 can also receive signals and communicate information associated with the received signals to an ICE catheter ultrasound processing system via the cable connection 110. As an example of its dimensions, the flexible circuit 105, when separated from the test connection area 101 (e.g., by cutting the flexible circuit at line 121) is sized for insertion into a vessel of a patient as part of an ICE catheter. As used herein, “flexible” can generally refer to the ability (e.g., of a material or an article) to bend freely and repeatably without breaking and/or to conform to the shape of the body part to which the flexible material (or article) is applied. The flexible circuit 105 can have an outer surface 102 and an inner surface 103 (see, for example, FIGS. 2A and 2B) which can be configured to face an opposite direction of the outer surface 102. Additionally, in some embodiments, the flexible circuit 105 can have a conductive layer. The conductive layer can be disposed between the outer surface 102 and the inner surface 103. The flexible circuit 105 can also have a cover layer located at or near the inner surface 103 of the flexible circuit 105. In some embodiments, the flexible circuit 105 can have a base layer located at or near the outer surface 102. An electrical connection between a component and traces or other conductive features of the conductive layer can be made through a “cutout” or “hole” in the cover layer. For example, transducers of the transducer array can be connected to traces through cutouts. In an example, connectors or cables can be connected to traces through cutouts.

A transducer array 108 can be coupled to a proximal region 106 of the flexible circuit 105, and when the transducer array 108 is coupled to the flexible circuit 105, the transducer array 108 can be considered to be a part of the flexible circuit 105. As illustrated in FIGS. 1A and 1B, the flexible circuit 105 can also have a cable connection portion 110 that includes electrical connectors 111 that are formed in the conductive layer and connected to the traces 112 that are connected to each transducer of the transducer array 108 when the transducer array 108 is attached to the flexible circuit 105. The electrical connectors 111 can each be connected to one of a plurality of electrical lines (e.g., bundles 114, cables) to electrically connect to the transducer array 108, as described further in reference to FIGS. 2A-2B.

The flexible circuit 105 can also have a test connection area 101. The test connection area 101 is structured to have an electrical connection to each trace that is formed in the flexible circuit 105 and connected to one of the plurality of transducers of the transducer array 108. The test connection area 101 is configured to be wider than the cable connection portion 110 to advantageously allow test equipment to be attached to the flexible circuit for testing because of the larger (wider) electrical interface. For example, the test connection arca 101 can be used for testing connectivity of the traces of the flexible circuit, and/or for testing of the connectivity of traces and transducers of the transducer array. In some embodiments, the test connection area 101 is between 2 times and 4 times as wide as the cable connection portion 110. In various embodiments, the test connection area 101 allows for test equipment to be attached to the test connection area and determine whether the transducer array 108 and cable connection portion 110 are successfully coupled (e.g., electrically connected), if the ICE catheter device 100 is conductive, and if the ICE catheter device 100 can successfully relay a signal. If successfully connected, the test connection area 101 can be separated from the transducer array 108 and the cable connection portion 110 via a cut line 121. After determining the transducer array 108 and cable connection portion 110 are successfully connected, a user separates (e.g., cuts, removes) the test connection area 101 from the transducer array 108 and cable connection area at approximately the cut line 121. The transducer array 108 and the cable connection portion can be incorporated on an ICE catheter, for example, on a distal (e.g., tip) of an ICE catheter

FIG. 1B illustrates a partial top view of the flexible circuit intracardiac echocardiography (“ICE”) catheter device distal to the cut line 121 according to some embodiments. As shown in FIG. 1B, the flexible circuit 105 can have a distal region 104 and a proximal region 106. In some embodiments, the transducer array 108 can be positioned on the outer surface 102 of the proximal region 106 of the flexible circuit 105. The cable connection portion 110 is an area of the flexible circuit 105 structured for connecting the plurality of traces 112 to respective wires at connectors 111. In some embodiments, a number of wires (e.g., cables) can be formed into a bundle 114 (e.g., a bundle of wires, bundles of cables), each bundle 114 is connected (e.g., electrically and mechanically) to a connector 111 (Sec, for example, FIG. 2A). In other words, each wire in a bundle 114 can be connected to one of the plurality of traces at a connector 111. Each of one or more bundles 114 can be positioned on the outer surface 102 of the distal region 104 of the flexible circuit 105, located in the cable connection portion 110. Advantageously, the cable connection portion 110 has one or more cutouts 116 in a cover layer of the flexible circuit 105 which permit the cable connection portion 110 to be coupled to the plurality of traces 112 and relay signals to and from the transducer array 108.

As illustrated in FIG. 1B, there can be a distance 117 from the transducer array 108 to the cut line 121. In some embodiments, the distance 117 from the transducer array 108 to the cut line 121 is about 50 micrometers. In some embodiments, the distance 117 from the transducer array 108 to the cut line 121 can be between about 25 micrometers and about 75 micrometers. There is also a length 118 between the transducer array 108 and the cable connection portion 110. Advantageously, optimizing the length 118 between the transducer array 108 and the cable connection portion 110 can reduce the time it takes to relay signals between the transducer array 108 and the cable connection portion 110. In some embodiments, the length 118 between the transducer array 108 and the cable connection portion 110 can be approximately 0.913 mm. However, in some embodiments, the length 118 between the transducer array 108 and the cable connection portion 110 can be between 0.5 mm and 1 mm. Additionally, the length 119 of the cable connection portion 110 can be 2 mm. Optimizing the length 119 of the cable connection portion 110 can reduce (e.g., shorten) the length of the plurality of traces 112 to improve the signal processing time between the transducer array 108 and the cable connection portion 110. Additionally, the length 119 of the cable connection portion 110 can be between 1 mm and 3 mm to improve signal processing time based on the requirements of the ICE catheter device 100. Additionally, the cable connection portion 110 can have a width 125 of approximately 56.91 mm. In some embodiments, the width 125 can be between 50 mm and 70 mm.

The transducer array 108 can be coupled (e.g., electrically connected) to a plurality of traces 112, and the plurality of traces 112 can be configured to couple the transducer array 108 to the cable connection portion 110 and one or more connectors 111. The path of the plurality of traces 112 which can couple the transducer array 108 and the cable connection portion 110 can follow or extend along a non-linear path. In some embodiments, the plurality of traces 112 can be made of copper, however different materials can be used for the plurality of traces 112 (e.g., silver, annealed copper, gold, aluminum, calcium, tungsten, zinc, etc.). Furthermore, the plurality of traces 112 can connect to the one or more connectors 111 located at the cable connection portion 110. The transducer array 108 can comprise a plurality of transducers 109. The plurality of transducers 109 can emit a signal which is capable of imaging portions of bodily tissues (e.g., heart, lungs). The plurality of transducers 109 can have a distal end 126 and a proximal end 127. When attached, the distal end 126 of the plurality of transducers 109 are coupled to the flexible circuit 105 at electrical connectors 122. The electrical connectors 122 are additionally connected to the transducer array 108. In some embodiments, the plurality of transducers 109 can be sixty-four transducers, where the sixty-four transducers are coupled to one the plurality of traces 112. Each of the sixty-four transducers can be individually coupled to the plurality of traces 112, where the plurality of traces 112 can be sixty-four traces. However, in some embodiments, the number of transducers can be sixteen to sixty-four (or more) transducers and the number of traces 112 can be 32 to sixty-four (or more) traces.

In some configurations of the flexible circuit, a subset of the plurality of traces 112 can connect to a portion (e.g., a left-side portion relative to the orientation in FIG. 1B) of the one or more connectors 111. Additionally, a subset of the plurality of traces 112 can connect to a portion (e.g., a right-side portion relative to the orientation in FIG. 1B) of the one or more connectors 111. The path of the plurality of traces 112 from the transducer array 108 to the cable connection portion 110 can be a non-linear path. Advantageously, this can allow the length of the plurality of traces 112 to be reduced to a minimal or optimal amount to reduce signal processing times. Ideally, for greater imaging, the time required for the signal to travel from the transducer array 108 to the cable connection portion 110 is minimized to ensure the signal received is not distorted or compromised during transmission. Therefore, by reducing the length between the transducer array 108 and the cable connection portion 110 with a non-linear arraignment of the plurality of traces 112, the signal can be significantly improved. To help enable this arrangement of the plurality of traces 112, each of the plurality of traces 112 can have a thickness of about, or exactly, 0.05 mm. In some embodiments, the shortest length of one of the plurality of traces 112 is about, or exactly, 1.40 mm. Additionally, the longest length of one of the plurality of traces 112 can be about, or exactly, 3.43 mm. Therefore, the range of lengths of the plurality of traces 112 can be between about 1.40 mm and about 3.43 mm with a percentage difference between the shortest trace and longest trace being approximately 41%. Advantageously, the minimal difference in the lengths of the plurality of traces 112 can improve the signal processing time since in an ideal scenario the percentage difference in lengths of the plurality of traces 112 would be approximately zero.

In some embodiments, the flexible circuit 105 can have a ground bar 120 which can surround the transducer array 108, the plurality of transducers 109, the cable connection portion 110 and the one or more connectors 111. The thickness of the ground bar 120 can be approximately 0.254 mm.

Referring to FIG. 1B, in some embodiments the one or more connectors 111 can be four connectors (e.g., 111a, 111b, 111c, 111d). Therefore, as shown in the illustrated embodiment, when the plurality of traces 112 are sixty-four traces, sixteen of the plurality of traces can go to each of the four connectors 111 so that each connector receives sixteen traces. In this example, at each connector 111a, 111b, 111c, 111d a subset of twelve of the traces can connect to a first side (e.g., a right side) of the connector and the subset including the remaining four traces can connect to an opposite side (e.g., a second side, or a left side) of the connector. As shown in FIG. 1B, all four of the connectors can have similar, if not identical, arrangements. However, in some embodiments, a subset of twelve of the traces can connect to the left side of the one or more connectors 111 and the subset of the remaining four traces can connect to a right side of one of the one or more connectors 111. Additionally, in some embodiments, eight of the traces can connect to the right side of the one or more connectors 111 and eight of the traces can connect to the left side of the one or more connectors 111. Other configurations of traces leading into a connector 111 are also contemplated.

In some embodiments, the flexible circuit 105 can have one or more cutouts 116 in the cover layer located on or through the flexible circuit 105. The one or more cutouts 116 can be configured to allow for coupling (e.g., electrically, mechanically) of different components in the ICE catheter device 100. The one or more cutouts 116 can be located inferior to the transducer array 108 when the transducer array 108 is coupled to the flexible circuit 105. In some embodiments, the one or more cutouts 116 can enable the transducer array 108 to be coupled to one or more backing blocks 113 (see, for example, FIGS. 2A-2B). The one or more backing blocks 113 can ensure that there is material between the transducer array 108 and the cable connection portion 110 when the transducer array 108 is rolled (e.g., folded) at a position distal to the transducer array 108 (see, for example, FIG. 2A-2B). Advantageously, the one or more cutouts 116 allow the plurality of traces 112 to couple to the transducer array 108 through the one or more cutouts 116. In some embodiments, the one or more cutouts 116 can allow the plurality of traces 112 to couple to the transducer array 108 disposed on another layer of the flexible circuit 105 (e.g., outer surface 102, base layer).

FIGS. 2A and 2B illustrate an end view and a perspective view of the ICE catheter device 100, where the catheter device is rolled (e.g., folded) at a region between the transducer array 108 and the cable connection portion 110. As illustrated in FIGS. 2A-2B, the ICE catheter device 100 can further comprise one or more bundles 114 which can be positioned at the cable connection portion 110. The cable connection portion 110 can be electrically connected to one or more bundles 114 which run perpendicular to the transducer array 108. Each bundle can include one or more electrical (signal) communication channels. For example, one or more wires. The one or more bundles 114 can be located inferior to the transducer array 108 in the folded configuration. Each of the one or more bundles 114 can be coupled to a respective one of the connectors 111 in the cable connection portion 110. When assembled for operation, the one or more bundles 114 are coupled to the connectors 111, and the connectors 111 are coupled to the plurality of traces 112. Advantageously, the arrangement of the plurality of traces 112 (see, for example, FIG. 1B) can reduce the signal time from the plurality of transducers 109 at the transducer array 108 to the one or more bundles 114 at the cable connection area 110. In some embodiments, the one or more bundles 114 can be coupled to an image generating device. Therefore, in some embodiments, signals emitted from the plurality of transducers 109 can travel from the plurality of transducers 109, to the cable connection portion 110, to the one or more bundles 114 and to the image generating device which can image a vessel or tissue structure (e.g., blood flow circulation, blood flow stoppage, etc.).

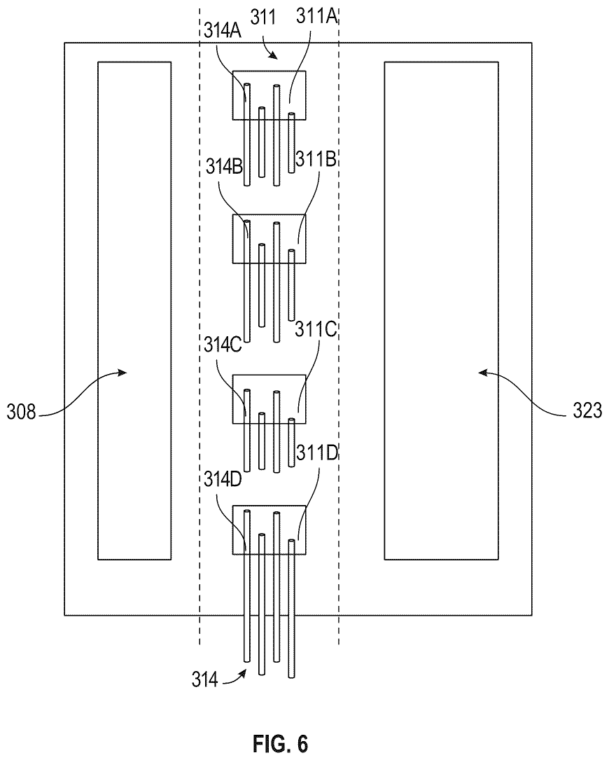

The one or more bundles 114 can be, in some embodiments, sixteen bundles where the sixteen bundles are coupled to the one or more connectors 111. Additionally, one of the one or more connectors 111 (e.g., connector 111A) can couple directly to four of the one or more bundles 114 (e.g., bundle 114A) when the one or more bundles 114 are sixteen bundles (see, for example, FIG. 6). In some embodiments, the one or more bundles 114 can have a diameter of approximately 185 micrometers.

In some embodiments, the flexible circuit 105 can have an inner surface 103 with one or more backing blocks 113 positioned opposite the transducer array 108. The one or more backing blocks 113 can be positioned above the one or more cables by folding the distal region 104 of the flexible circuit 105 underneath the transducer array 108.

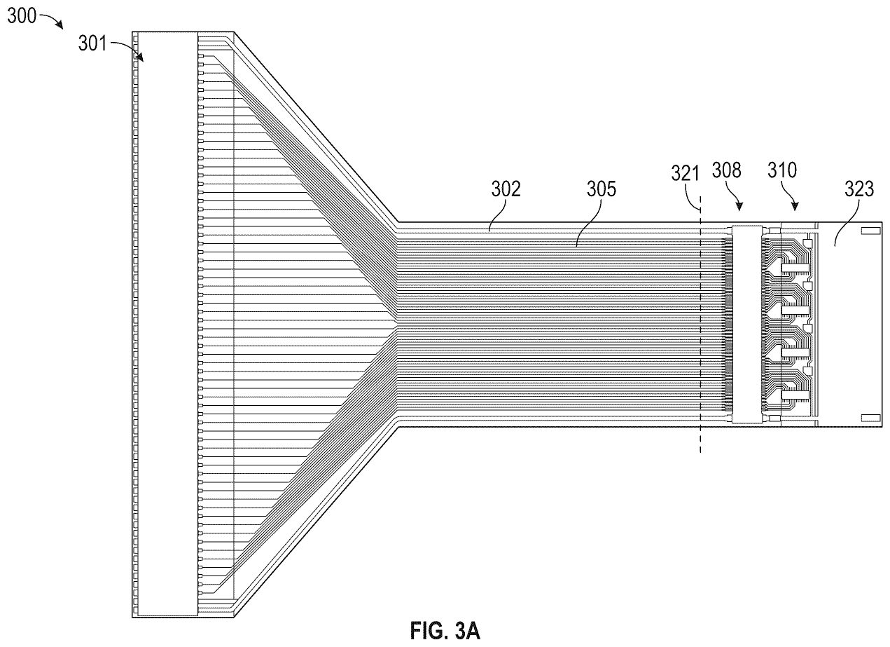

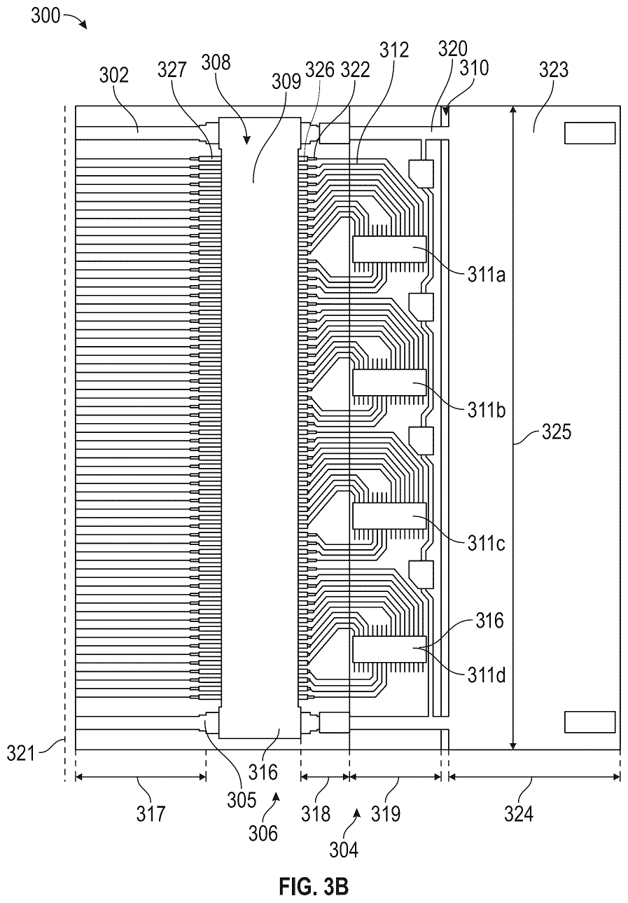

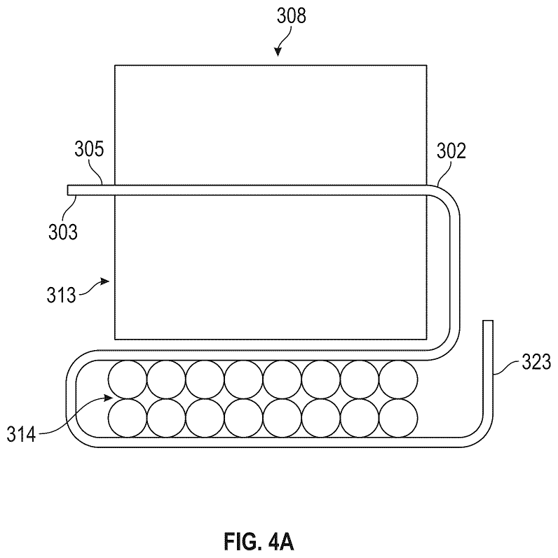

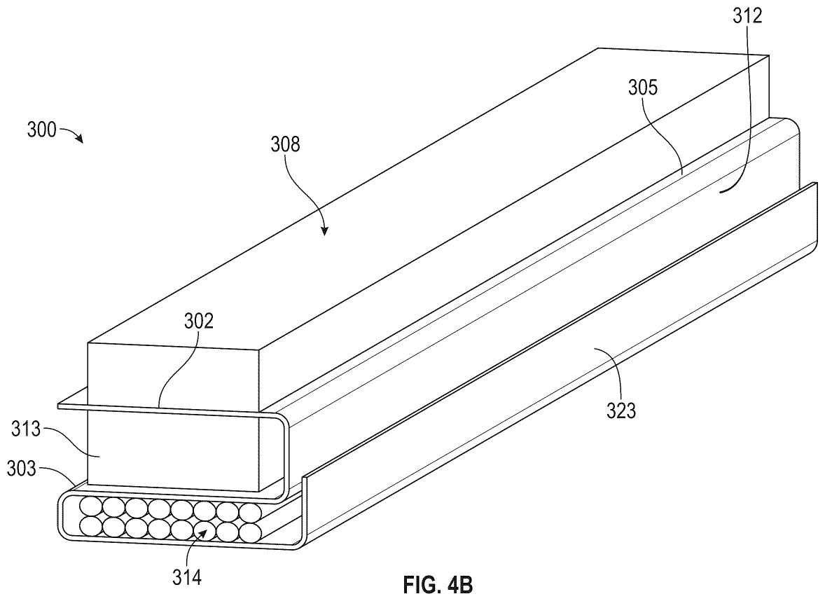

FIG. 3A illustrates a top view of another embodiment of an intracardiac echocardiography (“ICE”) catheter device 300 in a flat configuration. In many aspects, the ICE catheter device 300 is similar to the ICE catheter device 100 described above and the same or similar reference numbers are used to refer to the same or similar features. Therefore, the structure and description for the various features of the ICE catheter device 100 and how it's operated and controlled in FIGS. 1A-2B are understood to also apply to the corresponding features of the ICE catheter device 300 in FIGS. 3A-4B, except as described below. For example, the ICE catheter device 300 can be used to emit signals to and from a transducer array 308 to a cable connection portion 310.

As shown in FIG. 3A, the ICE catheter device 300 can include a flexible circuit 305 sized for insertion into a vessel of a patient. The flexible circuit 305 can have an outer surface 302 and an inner surface 303 which can be configured to face an opposite direction of the outer surface 302. Additionally, the flexible circuit 305 includes a conductive layer. The conductive layer is disposed between the outer surface 302 and the inner surface 303. The flexible circuit 305 can also have a cover layer located at or near the inner surface 303 of the flexible circuit 305. In some embodiments, the flexible circuit 305 can have a base layer located at or near the outer surface 302. The flexible circuit 305 can have a test connection area 301 at the most proximal edge of the ICE catheter device 300. Additionally, the flexible circuit 305 can have a transducer array 308, a cable connection portion 310, and a shield 323. The transducer array 308 and the cable connection portion 310 can be coupled (e.g., electrically connected). The shield 323 can be located distal to both the cable connection portion 310 and the transducer array 308. In some embodiments disclosed herein, the shield 323 can be used to protect components or areas (e.g., cable connection portion 310) of the ICE catheter device 300 (see for example, FIGS. 4A-4B). Advantageously, the test connection area 301 can determine whether the transducer array 308 and cable connection portion 310 are successfully coupled (e.g., electrically connected), if the ICE catheter device 300 is conductive, and if the ICE catheter device 300 can successfully relay a signal. If successfully connected, the test connection area 301 can be separated from the transducer array 308 and the cable connection portion 310 via a cut line 321. The cut line 321 can reduce the length of the ICE catheter device 300 when, after determining the transducer array 308 and cable connection portion 310 are successfully connected, a user separates (e.g., cuts, removes) the test connection area 301 from the transducer array 308 and cable connection portion 310 at the cut line 321.

FIG. 3B illustrates a partial top view of the intracardiac echocardiography (“ICE”) catheter device distal to the cut line 321 in a flat configuration according to an embodiment of the present disclosure. As shown in FIG. 3B, the flexible circuit 305 can have a distal region 304 and a proximal region 306. In some embodiments, the transducer array 308 can be positioned on the outer surface 302 of the proximal region 306 of the flexible circuit 305. Additionally, the cable connection portion 310 can be positioned on the outer surface 302 at the distal region 304 of the flexible circuit 305. The cable connection portion 310 can have one or more connectors 311. The cable connection portion 310 can be coupled to one or more connectors 311 (e.g., electrically or mechanically) to assist a relay of signals between the transducer array 308 and the cable connection portion 310. Advantageously, the cable connection portion 310 can have one or more cutouts 316 located in the cover layer which can permit the cable connection portion 310 to relay signals to and from the transducer array 308 through a plurality of traces 312 located in the conductive layer of the flexible circuit 305.

As illustrated in FIG. 3B, there is a distance 317 from the transducer array 308 to the cut line 321. In some embodiments, the distance from the transducer array 308 to the cut line 321 is 50 micrometers. However, the distance 317 from the transducer array 308 to the cut line 321 can be between 25 micrometers and 75 micrometers. There is also a length 318 between the transducer array 308 and the cable connection portion 310. Advantageously, the length 318 between the transducer array 308 and the cable connection portion 310 can reduce the signal relay time between the transducer array 308 and the cable connection portion 310. In some embodiments, the length 318 between the transducer array 308 and the cable connection portion 310 can be 0.913 mm. However, in some embodiments, the length 318 between the transducer array 308 and the cable connection portion 310 can be between 0.5 mm and 1 mm. Additionally, the length 319 of the cable connection portion 310 can be 2 mm. Advantageously, reducing the length of the traces can improve the signal processing time between the transducer array 308 and the cable connection portion 310. However, in some embodiments, the length of the cable connection portion 310 can be between 1 mm and 3 mm.

The transducer array 308 can be coupled (e.g., electrically connected) to a plurality of traces 312. The plurality of traces 312 can be configured to couple the transducer array 308 to the cable connection portion 310. The path of the plurality of traces 312 coupling the transducer array 308 and the cable connection portion 310 can follow (or extend along) a non-linear path. In some embodiments, the plurality of traces 312 can be made of copper, however different materials can be used for the plurality of traces 312 (e.g., silver, annealed copper, gold, aluminum, calcium, tungsten, zinc, etc.). Furthermore, the plurality of traces 312 can connect to the one or more connectors 311 located at the cable connection portion 310. The transducer array 308 can comprise a plurality of transducers 309, where the plurality of transducers 309 can emit a signal which is capable of imaging portions of bodily vessels or tissues (e.g., heart, lungs). The plurality of transducers 309 can have a distal end 326 and a proximal end 327, the distal end 326 of the plurality of transducers 309 can be coupled to an electrical connector 322 (e.g., a plurality of electrical connectors 322). The plurality of traces 312 can be coupled to a plurality of electrical connectors 322, where the plurality of electrical connectors 322 are additionally connected to the transducer array 308. In some embodiments, the plurality of transducers 309 can be sixty-four transducers, where the sixty-four transducers can be coupled to the plurality of traces 312. Each of the sixty-four connectors can be individually coupled to the plurality of traces 312, where the plurality of traces 312 can be sixty-four traces. However, in some embodiments, the number of transducers can be thirty-two to sixty-four transducers and the number of traces can be thirty-two to sixty-four traces.

In some embodiments, a subset of the plurality of traces 312 can connect to a left portion of the one or more connectors 311. Additionally, a subset of the plurality of traces 312 can connect to a right portion of the one or more connectors 311. As illustrated in FIG. 3B, the path of the plurality of traces 312 from the transducer array 308 to the cable connection portion 310 can be a non-linear path. Advantageously, the non-linear path can allow the length of the plurality of traces 312 to be reduced to a minimal amount to reduce signal processing times. Ideally, for greater imaging, the signal from the transducer array 308 to the cable connection portion 310 would be minimized to ensure the signal received is not distorted or compromised while being transmitted. Therefore, by reducing the length between the transducer array 308 and the cable connection portion 310 (e.g., by incorporating a non-linear arraignment of traces), the signal can be significantly improved. To help enable this arrangement of the plurality of traces 312, each of the plurality of traces 312 can have a thickness of 0.05 mm. In some embodiments, the shortest length of one of the plurality of traces 312 is 1.40 mm. Additionally, the longest length of one of the plurality of traces 312 can be 3.43 mm. Therefore, the range of lengths of the plurality of traces 312 can be between 1.40 mm and 3.43 mm with a percentage difference between the shortest trace and longest trace being approximately 41%. Advantageously, the minimal difference in the lengths of the plurality of traces 312 can improve the signal processing time (e.g., since in an ideal scenario the percentage difference in lengths of the plurality of traces 312 would be approximately zero).

In some embodiments, the flexible circuit 305 can comprise a ground bar 320 which can surround the transducer array 308, the plurality of transducers 309, the cable connection portion 310 and the one or more connectors 311. The thickness of the ground bar 320 can be approximately 0.254 mm. Additionally, the ground bar 320 can feature cutouts along the ground bar 320.

As illustrated in FIG. 3B, the one or more connectors 311 can be four connectors (e.g., 311a, 311b, 311c, 311d). Therefore, in embodiments where the plurality of traces 312 are sixty-four traces, sixteen of the plurality of traces 312 can go to each of the four connectors so that each connector has sixteen traces. At one connector, a subset of twelve of the traces can connect to the right side of one of the connectors and the subset of the remaining four traces can connect to a left side of the one connector. As shown in FIG. 3B, all four of the connectors can have similar, if not identical, arrangements. However, in some embodiments, a subset of twelve of the traces can connect to the left side of one of the connectors and the subset of the remaining four traces can connect to a right side of the one connector. Additionally, in some embodiments, eight of the traces can connect to the right side of the connector and eight of the traces can connect to the left side of the connector.

In some embodiments, the flexible circuit 305 can have one or more cutouts 316 which can be in the cover layer on or through the flexible circuit 305. The one or more cutouts 316 can be configured to allow for coupling (e.g., electrically, mechanically) of different components in the ICE catheter device 300. The one or more cutouts 316 can be located inferior to the transducer array 308. In some embodiments, the one or more cutouts 316 can enable the transducer array 308 to be coupled to one or more backing blocks 313 (see, for example, FIGS. 4A-4B). The one or more backing blocks 313 ensure that there is material between the transducer array 308 and the cable connection portion 310 when the transducer array 308 is rolled (e.g., folded) at a position distal to the transducer array 308 (see, for example, FIG. 4A). Advantageously, the one or more cutouts 316 can allow the plurality of traces 312 to couple to the transducer array 108 through the one or more cutouts 316. In some embodiments, the one or more cutouts 316 can allow the plurality of traces 312 to couple to the transducer array 308 disposed on another layer of the flexible circuit 305 (e.g., outer surface 302, base layer).

As illustrated in FIGS. 3A and 3B, the ICE catheter device 300 can have a shield 323, where the shield 323 is located distal to the cable connection portion 310. In some embodiments, the shield 323 can be made of copper. However, the shield 323 can also be made of other materials (e.g., silver, annealed copper, gold, aluminum, calcium, tungsten, zinc, etc.). The shield 323 can have a length 324 of approximately 25.028 mm. In some embodiments, the shield 323 can have a length 324 between 22 mm to 28 mm. Additionally, the shield 323 can have a width 325 of approximately 56.91 mm. In some embodiments, the shield 323 can have a width 325 between 50 mm to 70 mm. Advantageously, in some embodiments the width 325 of the shield 323 can be equal to the width (i.e., width 125) of the cable connection portion 310 (see FIG. 1B for reference). Alternatively, the shield 323 width can be slightly less or slightly more than the width 125 of the cable connection portion 110.

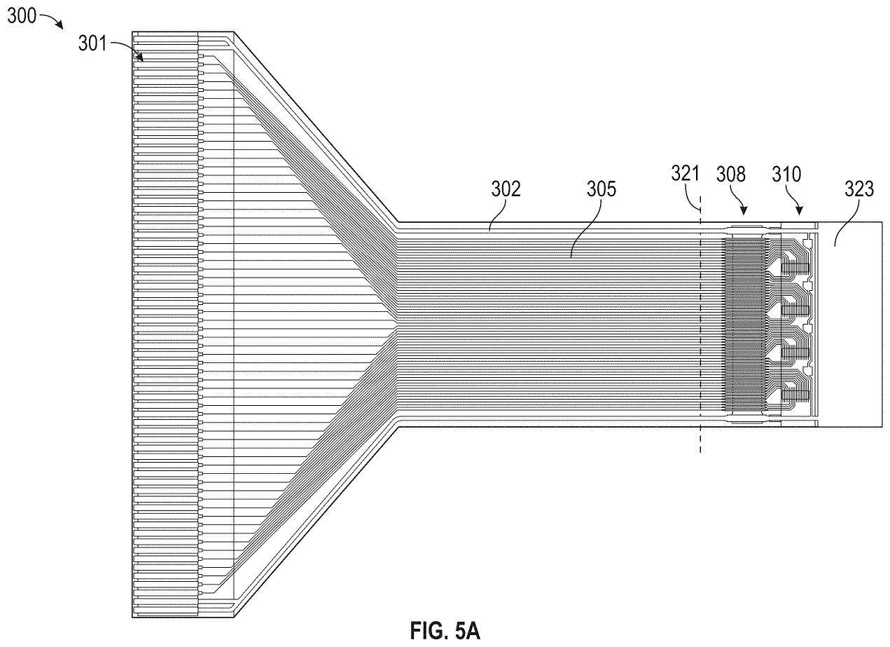

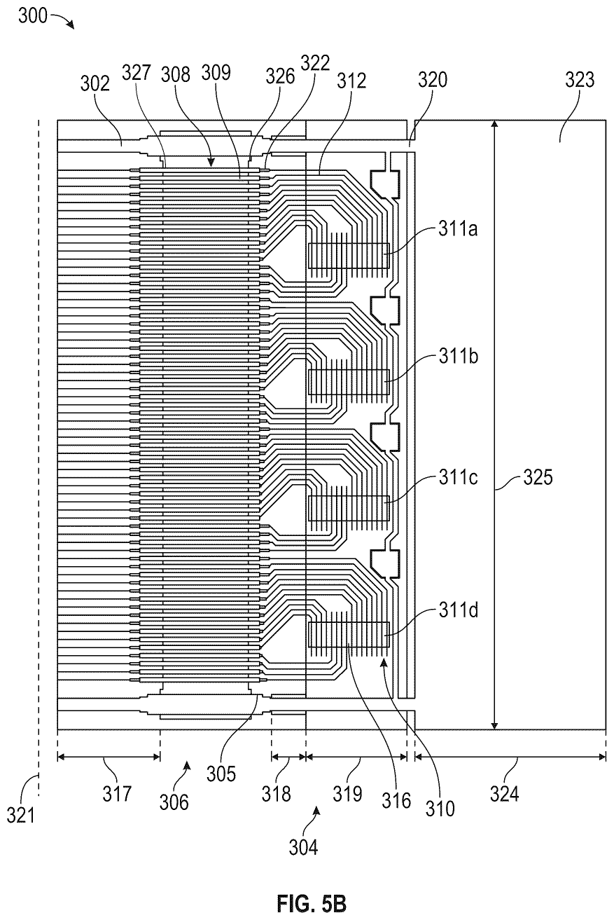

In some embodiments, the shield 323 can also have one or more cutouts 316, where the one or more cutouts 316 of the shield 323 can be located at or near the surface of the shield 323. Additionally, the shield 323 can have cutouts located inferior to the shield 323 on a distal portion of the flexible circuit 305 (see, for example, FIG. 5B).

FIGS. 4A and 4B illustrate an end view and a perspective view of the ICE catheter device 300, where the catheter device is rolled (e.g., folded) at a region between the transducer array 308 and the cable connection portion 310 and a region between the cable connection portion 310 and the shield 323. As illustrated in FIGS. 4A-4B, the ICE catheter device 300 can further have one or more bundles 314 which can be positioned on the cable connection portion 310. The cable connection portion 310 can have one or more bundles 314 which run perpendicular to the transducer array 308. The one or more bundles 314 can be located inferior to the transducer array 308. The one or more bundles 314 can be coupled to one or the one or more connectors 311, when the one or more connectors 311 are coupled (e.g., electrically, mechanically) to the cable connection portion 310, as illustrated in FIGS. 4A-4B. In some embodiments, the one or more bundles 314 can be configured to couple to the one or more connectors 311 when the one or more connectors 311 are coupled to the plurality of traces 312. Advantageously, the arrangement of the plurality of traces 312 (see, for example, FIG. 3B) can reduce the signal time between the plurality of transducers 309 at the transducer array 308 to the one or more bundles 314 at the cable connection portion 310. In some embodiments, the one or more bundles 314 can be coupled to an image generating device. Therefore, in some embodiments, signals emitted from the plurality of transducers 309 can travel from the plurality of transducers 309, to the cable connection portion 310, to the one or more bundles 314 and to the image generating device which can image a vessel or tissue structure (e.g., blood flow circulation, blood flow stoppage, etc.).

The one or more bundles 314 can be, in some embodiments, sixteen bundles where the sixteen bundles are coupled to the one or more connectors 311. The sixteen bundles can couple to the one or more connectors 311. Additionally, the one of the one or more connectors 311 can couple directly to four of the one or more bundles 314 when the one or more bundles 314 are sixteen bundles.

In some embodiments, the flexible circuit 305 can have an inner surface 303 with one or more backing blocks 313 positioned opposite the transducer array 308. The one or more backing blocks 313 can be positioned above the one or more cables by folding the distal region 304 of the flexible circuit 305 underneath the transducer array 308.

The shield 323 can advantageously protect the one or more bundles 314 when the flexible circuit 305 is rolled (e.g., folded) at a position distal to the cable connection portion 310 but proximal to the shield 323. The shield 323, in this embodiment, can ensure that the one or more bundles 314 or the cable connection portion 310 are not damaged by any fluids or substances that may interact with the area when the ICE catheter device 300 is within a tissue or vessel. Additionally, the shield 323 can be positioned to protect a lateral portion of the one or more bundles 314, as illustrated in FIGS. 4A-4B. Similarly, this position can prevent the cable connection portion 310 from being damaged by and fluids or substances within the tissue or vessel.

FIGS. 5A and 5B illustrate another embodiment of an intracardiac echocardiography (“ICE”) catheter device 500. In many aspects, the ICE catheter device 500 is similar to the ICE catheter device 100, 300 described above and the same or similar reference numbers are used to refer to the same or similar features. For example, the ICE catheter device 500 includes the test connection area 501, the outer surface 502, the inner surface 503, the distal region 504, the proximal region 506, the transducer array 508 and the cable connection portion 510 that are similar to corresponding features described above. Additionally, the ICE catheter device 500 includes a plurality of transducer 509, one or more connectors 511, a plurality of traces 512, a ground bar 520, and a plurality of electrical connectors 522 that are all similar or identical to the corresponding features described above.

As illustrated in FIGS. 5A and 5B the one or more cutouts 516 can be located inferior to the transducer array 508. Furthermore, the plurality of traces 512 and the shield 523 can be located superior to the one or more cutouts 516.

FIG. 6 illustrates the intracardiac echocardiography (“ICE”) catheter device 300 in an unfolded perspective view with one or more bundles 314 connected to one or more connectors 311. The one or more bundles 314 can include sixteen wires. The one or more connectors 311 can be four connectors (e.g., connector 311A, 311B, 311C, 311D). Each of the sixteen wires of the bundles 314 can be split into four groups (e.g., four bundles) of four wires (e.g. bundle 314A, 314B, 314C, 314D). The four groups of four wires (e.g. bundle 314A, 314B, 314C, 314D) can each connect to one of the corresponding four connectors (e.g., connector 311A, 311B, 311C, 311D. For example, bundle 314A (e.g., four wires) is coupled (e.g., electrically connected) to connector 311A. Additionally, the shield 323 is displaced distal to the one or more bundles 314 and the one or more connectors 311 (e.g., leftwards in FIG. 6). Although FIG. 6 is shown and described in connection with the ICE catheter device 300, one of skill in the art will recognize that the ICE catheter device 100 of FIGS. 1A-2B, and ICE catheter device 500 of FIGS. 5A-5B can also function as disclosed above.

FIG. 7 illustrates the intracardiac echocardiography (“ICE”) catheter device 100, where the flexible circuit 105 is in a rolled (e.g., folded) view and positioned on an insertion device 140 of the ICE catheter device 100. The insertion device 140 is configured to be inserted into vessel walls or tissue structure. The outer surface 102 of the flexible circuit 105 can be positioned on an exterior region of the insertion device 140. The transducer array 108 is positioned on the outer surface 102. The transducer array 108 is configured to transmit (e.g., emit) ultrasonic energy to create an image of the tissue or vessel. The signal from the transducer array 108 to the cable connection portion 110 and to the one or more bundles 114 (and vise-versa) can image the vessel where the transducer array 108 and/or insertion device 140 is placed. Although FIG. 7 is shown and described in connection with the ICE catheter device 100, one of skill in the art will recognize that the ICE catheter device 300 of FIGS. 3A-4B, and ICE catheter device 500 of FIGS. 5A-5B can also function as disclosed above.

In some embodiments, one or more methods of assembly for an intracardiac echocardiography (“ICE”) catheter device 100, 300 are disclosed. In many aspects, the method for assembling the ICE catheter device 300 is similar to the ICE catheter device 100 described above. Therefore, the structure and description for the various features of the ICE catheter device 100 and how it's operated and controlled in FIGS. 1A-2 are understood to also apply to the corresponding features of the device 300 in FIGS. 3A-5B, except as described below. The method can include obtaining a flexible circuit having an outer surface 102 and an inner surface 103, where the inner surface 103 can be opposite of the outer surface 102. The flexible circuit 105, in some embodiments, can have a distal region 104 and a proximal region 106. Further, the method can include positioning a transducer array 108 having a plurality of transducers 109 on the outer surface 102 of the proximal region 106 of the flexible circuit, positioning a cable connection portion 110 having one or more connectors 111 and one or more cutouts 116 on the distal region 104 of the flexible circuit 105, arranging a plurality of traces 112 on the flexible circuit 105 to couple the transducer array 108 to the cable connection portion 110, and coupling one or more bundles 114 to the one or more connectors 111. The one or more cutouts 116 in the cable connection portion 110 can permit the one or more bundles 114 to couple to the one or more connectors 111.

In some embodiments, the method can include positioning the one or more bundles 114 beneath the one or more backing blocks 113 by folding or rolling the distal region 104 of the flexible circuit 105 underneath the transducer array 108. Additionally, the flexible circuit 305 can have one or more backing blocks 113 and a shield 323. The method can further include positioning one or more backing blocks 313 on the inner surface 103 of the flexible circuit 105, which can be opposite of the transducer array 108. The shield 323 can be located on the distal region 304 of the flexible circuit 305 and distal to the cable connection portion 310. To position the one or more bundles 314 beneath the one or more backing blocks 313 the distal region 304 of the flexible circuit 305 can be rolled (e.g., folded) underneath the transducer array 308. Additionally, in some embodiments, the method can include folding the distal region 304 of the flexible circuit 305 at a location distal to the cable connection portion 310 and proximal to the shield 323. Advantageously, the shield 323 can cover a lateral portion of the one or more bundles 314.

### Additional Embodiments

In embodiments of the present disclosure, devices and methods for relieving pressure on a portion of a knee may be in accordance with any of the following clauses:

Clause 1. An intracardiac echocardiography (“ICE”) catheter device, comprising: a flexible circuit sized for insertion into a vessel of a patient, the flexible circuit comprising: an outer surface and an inner surface configured to face an opposite direction of the outer surface; a distal region; a proximal region having a plurality of traces; a transducer array positioned on the outer surface of the proximal region and electrically coupled to the plurality of traces, the plurality of traces arranged to electrically couple to a plurality of transducers of the transducer array; a backing block on the inner surface opposite the transducer array; a cable connection portion in the distal region, the cable connection portion having one or more connectors; one or more bundles positioned on the outer surface coupled to the one or more connectors in the cable connection portion, where each bundle includes a plurality of communication channels, wherein the cable connection portion is positioned such that a cable connection portion inner surface faces the backing block, and the backing block is between the transducer array and the cable connection portion.

Clause 2. The device of clause 1, wherein the flexible circuit comprises a shield located on a portion of the flexible circuit such that the cable connection portion is between the transducer array and the shield.

Clause 3. The device of clause 2, further wherein the shield is adjacent to the cable connection portion such shield is folded behind the cable connection portion and the outer surface of the shield faces the cable connection portion.

Clause 4. The device of clause 1, wherein the plurality of transducers comprises sixty-four transducers.

Clause 5. The device of clause 4, wherein the plurality of transducers are coupled to the plurality of traces, wherein the plurality of traces is sixty-four traces.

Clause 6. The device of clause 1, wherein the one or more connectors comprises four connectors.

Clause 7. The device of clause 1, wherein one of the one or more connectors are coupled to sixteen of the plurality of traces.

Clause 8. The device of clause 1, wherein the one or more bundles are sixteen bundles.

Clause 9. The device of clause 8, wherein one of the one or more connectors is coupled to four of the one or more bundles.

Clause 10. The device of clause 1, wherein the transducer array is distal to a cut line.

Clause 11. The of clause 10, wherein a distance from the cut line to the transducer array is 50 micrometers.

Clause 12. The device of clause 10, wherein the flexible circuit comprises a test connection portion, the test connection portion located on one side of the cut line and the transducer array is located on an opposite side of the cut line.

Clause 13. The device of clause 12, wherein the test connection portion includes an electrical connection to each of the plurality of transducers.

Clause 14. The device of clause 1, wherein the plurality of traces has a thickness of 0.05 mm.

Clause 15. The device of clause 1, wherein the plurality of traces has a length between 1.40 mm and 3.43 mm.

Clause 16. The device of clause 1, wherein the plurality of traces are made of copper. Clause 17. The device of clause 2, wherein the shield has a length between 23 mm to 27 mm.

Clause 18. The device of clause 17, wherein the shield has a width between 50 mm to 60 mm.

Clause 19. The device of clause 2, wherein the shield comprises one or more cutouts. Clause 20. The device of clause 2, wherein the shield is made of copper.

Clause 21. The device of clause 1, wherein the flexible circuit comprises a ground bar, wherein the ground bar is arranged to be at least partially around the plurality of transducers and the one or more connectors.

Clause 22. The device of clause 1, wherein a subset of the plurality of traces connects to a left portion of the one or more connectors.

Clause 23. The device of clause 1, wherein a subset of the plurality of traces connects to a right portion of the one or more connectors.

Clause 24. The device of clause 1, wherein a length between the transducer array and the cable connection portion is between 0.85 mm and 0.95 mm.

Clause 25. The device of clause 1, wherein the cable connection portion has a length of approximately 2 mm.

Clause 26. The device of clause 1, wherein the plurality of traces are coupled to a plurality of electrical connectors.

Clause 27. The device of clause 1, wherein an arraignment of the plurality of traces reduces a signal processing time from the one or more bundles to the plurality of transducers.

Clause 28. The device of clause 1, wherein one of the one or more bundles has a diameter of approximately 185 micrometers.

Clause 29. The device of clause 1, wherein the plurality of traces extend in a non-linear path from the transducer array to the one or more connectors.

Clause 30. The device of clause 29, wherein the non-linear path improves a signal processing time for the device.

Clause 31. A method of assembly of circuit for an intracardiac echocardiography (“ICE”) catheter device, comprising: obtaining a flexible circuit having an outer surface and an inner surface configured to face an opposite direction of the outer surface, a proximal portion for coupling to a transducer array, the proximal portion having a plurality of traces arranged to electrically couple to a plurality of transducers of the transducer array; a distal portion having a cable connection portion, the cable connection portion having one or more connectors electrically connected to the plurality of traces; a test connection area including an electrical connection to each of the plurality of traces; coupling the transducer array to the plurality of traces in the proximal portion; after testing an electrical connection between the transducer array and the plurality of traces using the test connection area, separating the test connection area from the proximal portion of the flexible circuit; and coupling one or more bundles positioned on the outer surface to the one or more connectors in the cable connection portion, where each bundle includes a plurality of communication channels.

Clause 32. The method of clause 31, further comprising positioning a backing block on the inner surface opposite the transducer array.

Clause 33. The method of clause 32, further comprising positioning the cable connection portion such that the inner surface of the cable connection portion faces the backing block.

Clause 34. The method of clause 33, wherein positioning the cable connection portion such that the inner surface of the cable connection portion faces the backing block includes folding a distal region of the flexible circuit underneath the transducer array.

Clause 35. The method of clause 31, wherein the flexible circuit comprises a shield located on a portion of the flexible circuit such that the cable connection portion is between the transducer array and the shield.

Clause 36. The method of clause 35, further comprising positioning a backing block on the inner surface opposite the transducer array.

Clause 37. The method of clause 36, further comprising positioning the cable connection portion such that the inner surface of the cable connection portion faces the backing block.

Clause 38. The method of clause 37 wherein positioning the cable connection portion such that the inner surface of the cable connection portion faces the backing block includes folding a distal region of the flexible circuit underneath the transducer array.

Clause 39. The method of clause 38, further comprising positioning the shield adjacent to the cable connection portion such shield is folded behind the cable connection portion such that the outer surface of the shield faces the cable connection portion.

Clause 40. A flexible circuit for positioning inside a catheter device, comprising: a transducer array having a plurality of transducer elements positioned in a first portion of the flexible circuit on a first surface; a plurality of traces arranged such that one of the plurality of traces electrically couples to each of the plurality of transducer elements on a first end and terminates at a connector at a second end in a second portion of the flexible circuit on the first surface; and wires connected to each of the plurality of traces at the connector, where the second portion is folded behind the first portion such that a first side of the first portion faces away from a first side of the second portion, and the wires connected to the plurality of traces are between the first portion of the flexible circuit and the second portion of the flexible circuit.

Clause 41. The flexible circuit of clause 40, wherein the catheter device is an intracardiac echocardiography (“ICE”) catheter device.

Clause 42. A flexible circuit for positioning inside a catheter device, comprising: a transducer array having a plurality of transducers positioned on an outer surface, a plurality of traces arranged to electrically couple to the plurality of transducers of the transducer array; a cable connection portion having one or more connectors; and a backing block on an inner surface; wherein the plurality of traces are configured to couple the cable connection portion to the transducer array.

Clause 43. The flexible circuit of clause 40, wherein the catheter device is an intracardiac echocardiography (“ICE”) catheter device.

Clause 44. A flexible circuit for positioning inside a catheter device, comprising: a test connection area; a transducer array; a plurality of traces on a flexible material; and a cable connection portion, the transducer array positioned between the test connection area and the cable connection portion, and wherein the plurality of traces is configured to connect the transducer array to the cable connection portion.

Clause 45. A flexible circuit for positioning inside a catheter device, comprising: a plurality of transducer elements arranged on a proximal section; a plurality of traces having a first end and a second end, wherein the first end is coupled to the plurality of transducer elements; a cable connection portion on a distal section; one or more backings opposite the plurality of transducer elements; and one or more bundles; wherein the one or more bundles are coupled to the cable connection portion; wherein the second end of the plurality of traces are coupled to the cable connection portion; wherein a signal is relayed from the plurality of transducer elements to the cable connection portion through the plurality of traces; wherein the distal section is folded underneath the proximal section.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

As is also stated above, it should be noted that the use of particular terminology when describing certain features or aspects should not be taken to imply that the terminology is being re-defined herein to be restricted to including any specific characteristics of the features or aspects of the technology with which that terminology is associated. Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The phrase “based on” does not mean “based only on,” unless expressly specified otherwise. In other words, the phrase “based on” describes both “based only on” and “based at least on.” Unless otherwise explicitly stated, articles such as “a” or “an” should generally be interpreted to include one or more described items. Accordingly, phrases such as “a device configured to” are intended to include one or more recited devices.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

The above description also discloses methods and materials of the present application. The ICE catheter device described herein may be susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. Applicant reserves the right to submit claims directed to combinations and sub-combinations of the disclosed inventions that are believed to be novel and non-obvious. Inventions embodied in other combinations and sub-combinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. An intracardiac echocardiography (“ICE”) catheter device, comprising:
a flexible circuit, comprising:
an outer surface and an inner surface configured to face an opposite direction of the outer surface;
a distal region;
a proximal region having a plurality of traces;
a transducer array positioned on the outer surface of the proximal region and electrically coupled to the plurality of traces, the plurality of traces arranged to electrically couple to a plurality of transducers of the transducer array;
a backing block on the inner surface opposite the transducer array;
a cable connection portion in the distal region, the cable connection portion having one or more connectors;
one or more bundles positioned on the outer surface coupled to the one or more connectors in the cable connection portion, where each bundle includes a plurality of communication channels, wherein the cable connection portion is positioned such that a cable connection portion inner surface faces the backing block, and the backing block is between the transducer array and the cable connection portion.

2. The device of claim 1, wherein the flexible circuit comprises a shield located on a portion of the flexible circuit such that the cable connection portion is between the transducer array and the shield.

3. The device of claim 2, wherein the shield is adjacent to the cable connection portion, wherein the shield is folded behind the cable connection portion and the outer surface of the shield faces the cable connection portion.

4. The device of claim 1, wherein each of the plurality of transducers are coupled to each of the plurality of traces, wherein the plurality of transducers include sixty-four transducers, wherein the plurality of traces include sixty-four traces.

5. The device of claim 4, wherein one of the one or more connectors are coupled to a group of sixteen traces of the sixty-four traces of the plurality of traces.

6. The device of claim 1, wherein the one or more bundles are sixteen wires, wherein one of the one or more connectors is coupled to four wires of the sixteen wires.

7. The device of claim 1, wherein the transducer array is distal to a cut line, wherein the flexible circuit comprises a test connection portion, wherein the test connection portion is located on one side of the cut line and the transducer array is located on an opposite side of the cut line, wherein the test connection portion includes an electrical connection to each of the plurality of transducers.

8. The device of claim 1, wherein the plurality of traces has a length between 1.40 mm and 3.43 mm.

9. The device of claim 1, wherein the flexible circuit comprises a ground bar, wherein the ground bar is arranged to be at least partially around the plurality of transducers and the one or more connectors.

10. The device of claim 1, wherein a subset of the plurality of traces connects to a left portion of the one or more connectors, wherein a second subset of the plurality of traces connects to a right portion of the one or more connectors.

11. The device of claim 1, wherein a length between the transducer array and the cable connection portion is between 0.85 mm and 0.95 mm, and wherein the cable connection portion has a length of approximately 2 mm.

12. The device of claim 1, wherein the plurality of traces are coupled to a plurality of electrical connectors.

13. The device of claim 1, wherein an arraignment of the plurality of traces reduces a signal processing time from the one or more bundles to the plurality of transducers, wherein the arrangement of the plurality of traces extend in a non-linear path from the transducer array to the one or more connectors to improve the signal processing time.

14. A method of assembly of circuit for an intracardiac echocardiography (“ICE”) catheter device, comprising:
obtaining a flexible circuit having
an outer surface and an inner surface configured to face an opposite direction of the outer surface,
a proximal portion for coupling to a transducer array, the proximal portion having a plurality of traces arranged to electrically couple to a plurality of transducers of the transducer array;
a distal portion having a cable connection portion, the cable connection portion having one or more connectors electrically connected to the plurality of traces;
a test connection area including an electrical connection to each of the plurality of traces;
coupling the transducer array to the plurality of traces in the proximal portion;
after testing an electrical connection between the transducer array and the plurality of traces using the test connection area, separating the test connection area from the proximal portion of the flexible circuit; and
coupling one or more bundles positioned on the outer surface to the one or more connectors in the cable connection portion, where each bundle includes a plurality of communication channels.

15. The method of claim 14, further comprising positioning a backing block on the inner surface opposite the transducer array and positioning the cable connection portion such that the inner surface of the cable connection portion faces the backing block, wherein positioning the cable connection portion such that the inner surface of the cable connection portion faces the backing block includes folding a distal region of the flexible circuit underneath the transducer array.

16. The method of claim 14, wherein the flexible circuit comprises a shield located on a portion of the flexible circuit such that the cable connection portion is between the transducer array and the shield.

17. The method of claim 16, further comprising positioning a backing block on the inner surface opposite the transducer array and positioning the cable connection portion such that the inner surface of the cable connection portion faces the backing block by folding a distal region of the flexible circuit underneath the transducer array.

18. The method of claim 17, further comprising positioning the shield adjacent to the cable connection portion such shield is folded behind the cable connection portion such that the outer surface of the shield faces the cable connection portion.

19. A flexible circuit for positioning inside a catheter device, comprising:
a transducer array having a plurality of transducer elements positioned in a first portion of the flexible circuit on a first surface;
a plurality of traces arranged such that one of the plurality of traces electrically couples to each of the plurality of transducer elements on a first end and terminates at a connector at a second end in a second portion of the flexible circuit on the first surface; and
wires connected to each of the plurality of traces at the connector,
where the second portion is folded behind the first portion such that a first side of the first portion faces away from a first side of the second portion, and the wires connected to the plurality of traces are between the first portion of the flexible circuit and the second portion of the flexible circuit.

20. The flexible circuit of claim 19, wherein the catheter device is part of an intracardiac echocardiography (“ICE”) catheter device.
